(19) United States
(12) Patent Application Publication
(10) Pub. No.: US 2020/0205857 A1
(43) Pub. Date: Jul. 2, 2020

(54) DEVICE FOR INTRA MEDULLARY ANTIBIOTICS PERFUSION

(71) Applicant: Akihiro MARUO, Himeji-shi, Hyogo (JP)

(72) Inventor: Akihiro MARUO, Himeji-shi, Hyogo (JP)

(21) Appl. No.: 16/089,951

(22) PCT Filed: Mar. 27, 2017
(86) PCT No.: PCT/JP2017/012279

§ 371 Date: Sep. 28, 2018

(30) Foreign Application Priority Data
Mar. 28, 2016 (JP) 2016-063474

(51) Int. Cl. A61B 17/34; A61B 17/60; A61B 17/16; A61M 39/10

(52) U.S. Cl. CPC A61B 17/3472; A61M 39/10; A61B 17/162; A61B 17/60

(57) ABSTRACT

A device for intra medullary antibiotic perfusion has a structure that can access easily from the outside and flexibly enables the administration of an antibiotic that is most suitable for a target site according to the circumstances.

The device for intra medullary antibiotics perfusion (iMAP) 100 is a device for perfusing an antibiotic into the bone fracture surgical site. It comprises a fixation pin (iMAP pin) 110 which comprises a shaft 111, a side face opening 114 formed on the side face of the shaft a base end opening 115 formed in the base end part of the shaft 111, a hollow portion 113 penetrating from the side face opening 113 to the base end opening 115 of the shaft and a screw part 112 formed besides the front end of the shaft 111 and to be screwed into a bone. The fixation pin (iMAP pin) 110 is fixing into a bone externally at a bone fracture site. A transfusion tube connector 120 receives an antibiotic provided from the antibiotic transfusion tube and allows intra medullary antibiotics perfusion from the side face opening 114 of the fixation pin (iMAP pin) 110 via the hollow portion 113.

### TECHNICAL FIELD

This invention relates to a device for intra medullary antibiotics perfusion (iMAP) that can perfuse antibiotics continuously to a patient's bone fracture surgical site for suppressing the occurrence of post traumatic deep surgical site infection (SSI). For example, iMAP can perfuse prescribed dose antibiotics continuously from outside to the patient's bone fracture surgical site like an intravenous drip.

### BACKGROUND ART

In an orthopedic field, it is very important to take care and suppress the occurrence of the post traumatic deep surgical site infection (SSI) and various complications. It is very important to keep on curing even after finishing the bone fracture surgical operation, especially in the case of the traumatic bone fracture with open wound fracture because there is a risk of the bacteria invasion into the tissue.

An effective treatment for suppressing the occurrence of post traumatic deep surgical site infection (SSI) is a local drug delivery of antibiotics to the patient's surgical site. However, the bone tissue and the peripheral tissue around the bone fracture surgical site do not readily respond to suppressing the occurrence of post traumatic deep surgical site infection (SSI). The reasons are described below.

1. The blood flow becomes poor because the bone tissue and the peripheral tissue are damaged by the huge force in the accident.

2. The blood and body fluid don't flow well and tend to stagnate at the damaged portion.

3. Antibiotics is hardly distributed to the patient's vascularized tissue by an intravenous drip method because the fine vascularized tissue is damaged too.

4. A bio-film (aggregation of bacteria) is formed around the metal tool for fixing the bone fracture surgical site. It becomes a barrier against antibiotic flow.

The method for delivering antibiotics should be selected by taking into consideration the above-mentioned circumstances.

In the prior art, there are 3 methods as the conventional antibiotics delivery methods.

1. Oral administration method=A preparation for internal use

2. Drip infusion method=Intravenous injection

3. Local delivery method=Indwell in the target portion by mixing antibiotics and bone cement

Even though the drip infusion method via the vascularized tissue can deliver the antibiotics to the target portion better compared with the oral administration method, but the antibiotics will be delivered around the whole body by either the oral administration method or the drip infusion method. Therefore, there is a limit on the dose level of the antibiotics. If the high dose antibiotics is delivered to the bone fracture treatment site by either the oral administration method or the drip infusion method, other organs may suffer adverse influence from being exposed to the high dose antibiotics delivered by the blood around the whole body. In contrast, the delivery range of antibiotics by the local delivery method is limited around the patient's therapeutic tissue, so the local delivery method has advantage of the dose level of the antibiotics having little influence on other organs.

In the prior art, there are several conventional inventions for perfusing antibiotics continuously to a patient's bone fracture surgical site as follows.

For example, JP 2015-509396 is known as shown by FIG. 18. This invention utilizes an implant tool to be embedded into the patient's therapeutic bone tissue in the bone fracture surgical site. It describes a system for perfusing the requested agent such as antibiotics by enclosing in the implant that provides a scaffold for bone regeneration and the base for perfusing the antibiotics. The portion where the implant is embedded is the bone fracture surgical site, so this invention can perfuse the antibiotics around the patient's therapeutic bone tissue.

For example, JP 2015-517879 is known as shown by FIG. 19. This invention utilizes a 3-dimensional implant. The 3-dimensional implant is manufactured by the multi-layer 3-dimensional printer with the applicable material for embedding into the bone tissue. The 3-dimensional implant is made of a permeable porous structure and has hollow space where the medical agent such as antibiotics and growth agent are contained. If the 3-dimensional implant is applied to the bone fracture surgical site, it can hold the antibiotics and perfuse the antibiotics around the bone fracture surgical site.

Prior art 1: JP 2015-509396

Prior art 2: JP 2015-517879

### DISCLOSURE OF THE INVENTION

### The Problems to be Solved

The conventional device for perfusing antibiotics in the prior art faces problems as follows.

The first problem is that the conventional device is an embedded type, so the indwell state in the target portion is maintained for a long term during the curing period. The dose level and the interval of antibiotics administration should be adjusted and changed according to the circumstances of the state of tissue. However, the conventional device is embedded into the target portion, so a subsequent operation is requested every time the antibiotics administration should be changed in its kind and its dose level in the embedded device. Moreover, the administration interval cannot be changed because the conventional device always perfuses antibiotics constantly.

The second problem is that the dose level adjustment is not possible even though the administration dose level perfused by the conventional device is gradually weakened. If the administration dose level is an appropriate dose level at the beginning of the embedding operation of the implant, the perfused dose level becomes gradually weaker. If the initial administration dose level of the antibiotics is adjusted higher to account for the weakened pace of the administration dose level, the initial administration of the antibiotics can cause an overdose, and the load on the patient becomes large.

As shown above problems, a new device is requested for providing a scaffold for perfusing antibiotics adjacent to and besides the bone fracture surgical site without indwelling in the target portion, which new device employs a structure accessible easily from the outside and able to perfuse appropriate antibiotics according to the circumstances of tissue in the bone fracture surgical site.

It is an object of the present invention to provide such a new device for providing a scaffold for perfusing antibiotics adjacent to and besides the bone fracture surgical site, which new device employs a structure accessible easily from the outside and able to perfuse appropriate antibiotics according to the status of the bone fracture surgical site according to the circumstances of tissue in the bone fracture surgical site. These characteristics are difficult to obtain by the conventional device in the prior art.

### Means for Solving the Problems

In order to achieve the above-mentioned object, the present invention of a device for intra medullary antibiotics perfusion (iMAP) for perfusing an antibiotics to a patient's bone fracture surgical site, applied as an external skeletal fixator comprises; a fixation pin for externally fixing to a bone by piercing from one side of cortical bone and penetrating through bone marrow to the other side of cortical bone; wherein the fixation pin includes a side face opening at the side of the shaft, which opening is located facing to the bone marrow; a base end opening at the base end part of the fixation pin; a hollow structure from the base end opening to the side face opening; and a male screw portion including spiral screw head mounted on the outer circumferential surface of the tip part of the fixation pin; wherein a perfusion point of antibiotics is limited to the side face opening of the shaft, there is no point in the male screw portion of the tip of the fixation pin, and antibiotics are perfused only into the bone marrow via the side face opening of the shaft.

According to the above-mentioned configuration, the present iMAP can provide a scaffold for perfusing antibiotics adjacent to and besides the bone fracture surgical site and can perfuse an appropriate kind and dose level of antibiotics according to the circumstances of tissue in the bone fracture therapeutic portion.

The present invention iMAP can provide an appropriate kind and dose level of antibiotics via a transfusion tube according to the circumstances of tissue in the bone fracture therapeutic portion.

At least, the iMAP has two phases for connecting to the external tools. The first phase is an electric drill connection phase for rotating and fixing the iMAP to the bone adjacent to and besides the bone fracture surgical site. In this first phase, the iMAP should be connected firmly to the drill chuck of the electric drill to rotate. The second phase is a transfusion tube connecting phase for perfusing antibiotics. In this second phase, the iMAP should be connected to the lock connector attached on the head of the transfusion tube.

As shown above, the present invention iMAP may connect to the external tools in these two phases. Therefore, there are two patterns for connecting procedures.

The first pattern is that a transfusion tube connector which shape can fit to the lock connector on the head portion of the external antibiotics transfusion tube is installed to the base end part of the iMAP, and the iMAP can connect to the antibiotics transfusion tube via the transfusion tube connector.

According to the first pattern, the transfusion tube connector is provided as a constant element attached to the iMAP, so the antibiotics transfusion tube can connect easily in the transfusion tube connecting phase.

In this first pattern, a drill attachment is included for providing a structure connecting to a drill chuck of an electric drill tool, which is used in the electric drill connecting phase.

The drill attachment comprises a transfusion tube adapter for connecting to the transfusion tube connector on the base end part of the fixation pin, wherein the adapter shape is the same as that of the lock connector on the head part of the transfusion tube, and a chuck connecter that can be chucked by the drill chuck of the electric drill tool.

By utilizing the drill attachment, the shape of the base end part of the fixation pin can be converted to the shape applicable to the electric drill tool by the drill attachment.

The second pattern is that a drill attachment is included for providing a structure connecting to a drill chuck of an electric drill tool installed to the base end part of the iMAP, so the iMAP can connect to the external electric drill tool via the chuck of the electric drill tool.

According to the second pattern, the drill attachment is provided as a constant element attached to the iMAP, so the external electric drill tool can connect easily in the electric drill connecting phase.

In this second pattern, a transfusion tube attachment is included for providing a structure connecting to the lock connector of the external transfusion tube in the transfusion tube connecting phase.

The transfusion tube attachment comprises a drill connector adapter for connecting to the drill connector and a transfusion tube connector for connecting to the lock connector on the head part of the transfusion tube.

By utilizing the transfusion tube attachment, the shape of the base end part of the fixation pin can be converted to the shape applicable to the lock connector of the external transfusion tube.

It is preferable that the transfusion tube attachment includes a detachable mechanism which can adjust the gripping force between the inner surface of the drill connector adapter and the outer surface of the drill connector of the fixation pin.

For example, the detachable mechanism comprises a gripping lock ball installed facing to the outer surface of the drill connector of the fixation pin; an elastic actuator; a gripping force adjuster for adjusting the gripping force corresponding to the pressure generated between the gripping lock ball and the outer surface of the drill connector when sliding along a track by the elastic actuator; wherein the structure of the gripping force adjuster contacting to the gripping lock ball has a wedge-shape. By this configuration, the gripping force adjuster moves along the track by the elastic actuator, the gripping force to the drill connector of the fixation pin by the gripping lock ball becomes large by the action of the wedge-shape on the gripping lock ball.

Next, it is preferable that the outer shape of the fixation pin is the same as the outer shape of a conventional pin for an external skeletal fixation applied to patient's bone fracture surgical site. By this configuration, the iMAP can apply as the external skeletal fixator providing both an external skeletal fixation function and an intra medullary antibiotics perfusion function to the patient's bone fracture surgical site.

Next, it is preferable that the fixation pin has a wider portion whose diameter is larger than that of another portion. If the patient's bone fracture is a complicated bone fracture, plural fixation pins are fixed to plural points adjacent to and besides patient's bone fracture surgical site, plural fixation pins are combined as a cluster. The present iMAP can provide a bridge connector for connecting between plural fixation pins even if there is a variation in diameter. The bridge connector can connect easily because the fixation pin of the iMAP has a wider portion which diameter is larger than that of another portion.

Antibiotic agents can be perfused from at least one of the devices for intra medullary antibiotics perfusion among the cluster.

### Effect of the Invention

According to the device for intra medullary antibiotics perfusion (iMAP) of the present invention, the present invention iMAP can provide a scaffold for perfusing antibiotics adjacent to and besides the bone fracture therapeutic portion and can perfuse an appropriate kind and dose level of antibiotics according to the circumstances of the tissue in the bone fracture therapeutic portion.

### BRIEF DESCRIPTION OF THE DRAWINGS

FIG. 1 is a schematic view of the structure of the device for intra medullary antibiotics perfusion 100 (outer screw type).

FIG. 2 is a schematic view showing the lock connector 210 of the external transfusion tube 200 is connected to the transfusion tube connector 120 of the iMAP 100 of Embodiment 1.

FIG. 3 is a schematic view of the structure of the drill attachment 140 and view showing the drill attachment 140 is connected to the transfusion tube adapter 141 of the iMAP 100 of Embodiment 1.

FIG. 4 is a schematic view showing the iMAP 100 is connected to the drill chuck of an electric drill tool of Embodiment 1.

FIG. 5 is a schematic view showing the iMAP 100 is fixed by piercing to the bone adjacent to and besides the patient's bone fracture surgical site.

FIG. 6 is a schematic view showing the drill attachment 140 is detached and the transfusion tube 200 is connected to the transfusion tube connector 120.

FIG. 7 is a schematic view of the structure of the device for intra medullary antibiotics perfusion (iMAP) 100a of this Embodiment 2.

FIG. 8 is a schematic view showing the transfusion tube attachment 160 is connected to the drill connector 150 of iMAP 100a of Embodiment 2.

FIG. 9 is a schematic view of the structure of the transfusion tube attachment 160.

FIG. 10 is a schematic view showing the operation of the gripping force adjuster 165 and the taper structure 166.

FIG. 11 is a schematic view showing the attachment procedure of the transfusion tube attachment 160 connecting to iMAP 100a and connecting to the lock connector 210 of the transfusion tube 200.

FIG. 12 is a schematic view showing the iMAP 100a is connected to the drill chuck of an electric drill tool of Embodiment 2.

FIG. 13 is a schematic view showing the iMAP 100a is fixed by piercing to the bone adjacent to and besides the patient's bone fracture surgical site.

FIG. 14 is a schematic view showing the transfusion tube 200 is connected to the transfusion tube connector 162 of the transfusion tube attachment 160.

FIG. 15 is a schematic structure of the iMAP 100 of Embodiment 1 which employs a different part 130 having a different diameter.

FIG. 16 is schematic structure of the iMAP 100a of Embodiment 2 which employs a different part 130 having a different diameter.

FIG. 17 is a schematic view showing the iMAP 100 of embodiment 3 is used as both an iMAP and a conventional external fixator.

FIG. 18 is a schematic view of the structure of a device for antibiotics perfusion in the prior art shown in the JP 2015-509396.

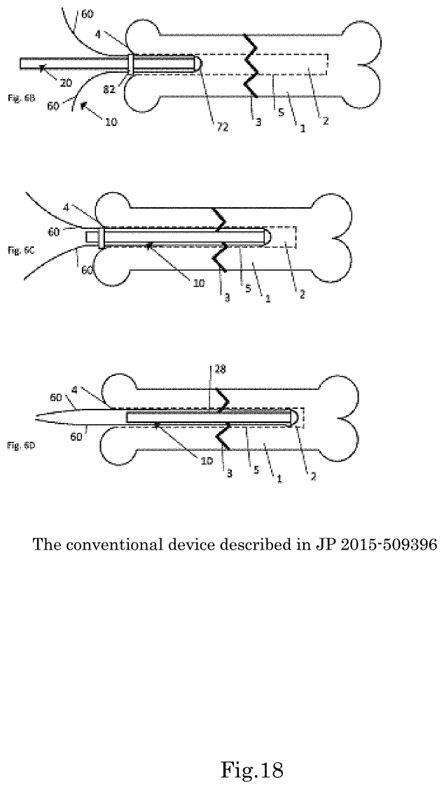

FIG. 19 is a schematic view of the structure of a device for antibiotics perfusion in the prior art shown in the JP 2015-517879.

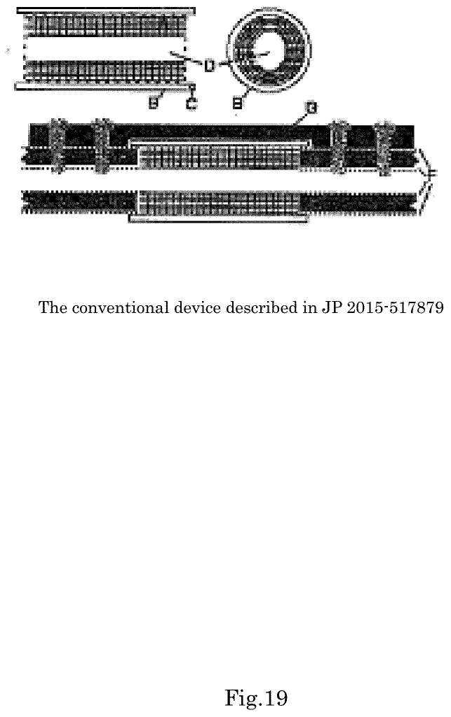

### DETAILED DESCRIPTION OF THE PREFERRED EMBODIMENT

Some embodiments of a device for intra medullary antibiotics perfusion according to the present invention are described below with reference to the relevant drawing. Needless to add, the claims of the present invention include but are not limited to the application, configuration, or quantity shown in the following embodiments.

Hereinafter, the configuration in which the fixation pin (iMAP pin) 110 and the transfusion tube connector 120 are combined as one body is described in Embodiment 1, the configuration in which the fixation pin (iMAP pin) 110 and the drill attachment 150 are combined as one body is described in Embodiment 2, and the example of the configuration applied to the an external skeletal fixation of the patient's bone fracture surgical site is described in Embodiment 3.

### Embodiment 1

The device for intra medullary antibiotics perfusion (iMAP) 100 of embodiment 1 according to the present invention is described with referencing to the drawings.

The procedure for applying iMAP has two phases for connecting to the external tools. The first phase is an electric drill connecting phase and the second phase is a transfusion tube connecting phase. In this Embodiment 1, the transfusion tube connector 120 used in the transfusion tube connecting phase is provided as a constant element, and the drill connector used in the electric drill connecting phase is provided as an attachment.

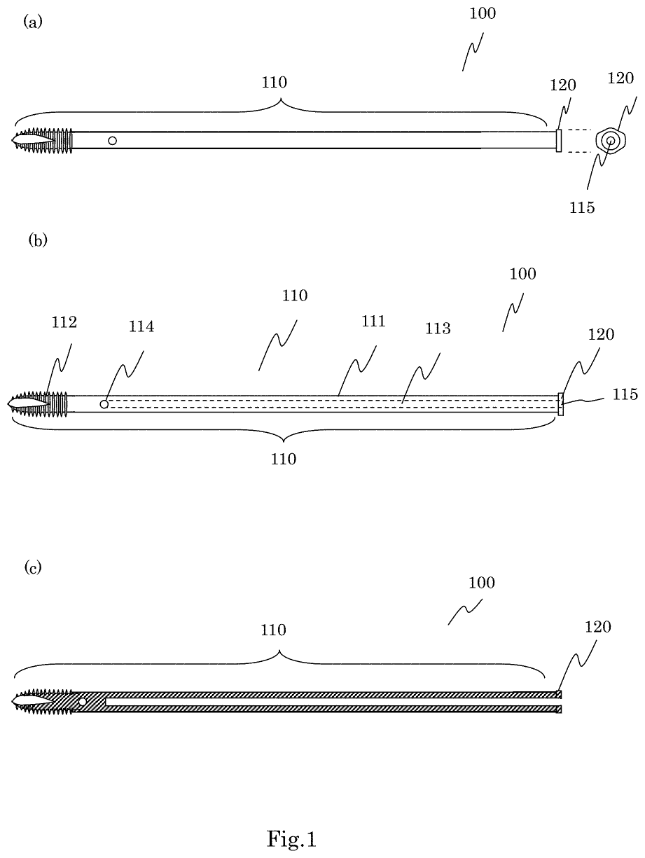

FIG. 1 is a schematic view of the structure of the device for intra medullary antibiotics perfusion (iMAP) 100.

As shown in FIG. 1 (a), the iMAP 100 includes a fixation pin (iMAP pin) 110, and a transfusion tube connector 120. FIG. 1 (b) is a figure for describing the elements of the iMAP 100. In FIG. 1 (b), a hollow portion 113 as an inner structure is shown by broken lines to recognize the inner structure easily.

In this Embodiment 1, the fixation pin (iMAP pin) 110 and the transfusion tube connector 120 are formed as one body and cannot be separated each other.

Each element of the fixation pin (iMAP pin) 110 is described below.

The fixation pin (iMAP pin) 110 is an element for externally fixing to a bone adjacent to and besides the bone fracture surgical site. The fixation pin (iMAP pin) 110 comprises a shaft 111, a screw part 112, a hollow portion 113, a side face opening 114 and a base end opening 115.

The shaft 111 is a rod shape pin made from stainless steel or titanium metal having a screw pin outer shape. The surface treatment with a hydroxyapatite coating is preferable for stable fixation.

The shaft diameter, whole length, and partial length of the screw part are varied according to the shape and the dimensions of the bone where the fixation pin (iMAP pin) 110 is applied. For example, the overall length is 150 mm, outer diameter is 5.0 mm, and partial length of the screw part is 15 mm. The base end shape is a stryker-Hoffmann type. It is provided as a disposable element.

The screw part 112 is a male screw portion including a spiral screw head mounted on the outer circumferential surface of the tip part of the fixation pin. The screw part 112 is provided as a portion to be screwed into the bone. The screw portion is varied in its pitch according to the shape and the dimensions of the bone where the fixation pin (iMAP pin) 110 is applied. For example, the male screw of this configuration has notched portion for assisting the fixation pin to screw into the harder bone tissue with reduced damage.

As the material and the shape for the shaft 111 and the screw part 112, the same material and the shape for the general fixation pin used as the external fixator for bone fracture can be employed. The general fixation pin used as the external fixator for bone fracture is widely known and generally used in orthopedics field for bone fracture treatment. The general fixation pin is a medical device whose operative method for fixing to the bone is established as the general external skeletal fixator. A lot of information about the general fixation pin is accumulated. It is convenient for the shaft 111 and the screw part 112 of the present invention iMAP to employ the same material and the shape for the general external fixator because the shaft 111 and the screw part 112 of the present invention iMAP is the same as the general fixation pin in view of the purpose of the fixing element for piercing into the bone adjacent to and besides the bone fracture surgical site.

Next, the hollow portion 113 is an inner space from the base end opening 11 to the side face opening 114. As described later, antibiotic is perfused through the hollow portion 113 to the patient's bone fracture surgical site. The inner shape of the hollow is not limited to particular shape. It is a simple cylindrical shape in this example.

The side face opening 114 is an opening on the side surface of the shaft 111 of the fixation pin (iMAP pin) 110 and is connected through the hollow portion 113. The side face opening 114 is located facing the bone marrow of the bone adjacent to and besides the bone fracture portion after fixing treatment. As described later, antibiotics is perfused to the patient's bone fracture surgical site only via the side face opening 114.

The base end opening 115 is located at the base end part of the fixation pin (iMAP pin) 110 and is connected through the hollow portion 113. As described later, the base end opening 115 works as an acceptable opening for antibiotics transfused from the external transfusion tube via the transfusion tube connector 120. In this configuration, the fixation pin (iMAP pin) 110 and the transfusion tube connector 120 are provided as one body, and the base end opening 115 is connected to the base end opening of the transfusion tube connector 120 directly.

It is preferable that the screw part 112, the hollow portion 113, the side face opening 114 and the base end opening 115 are made from stainless steel or titanium metal, and the surface treatment with a hydroxyapatite coating is provided for stable fixation.

The overall shape of the fixation pin (iMAP pin) 110 is the same as that of the general fixation pin used for the external skeletal fixator except for the original structure of the side face opening 114 on the side surface near the head portion and the base end opening 115 on the bottom end portion and hollow portion 113 through from the side face opening 114 to the base end opening 115.

Next, the transfusion tube connector 120 used in the phase for perfusing antibiotics is described below.

In the configuration of the iMAP 100 in Embodiment 1, the transfusion tube connector 120 is provided on the base end portion of the fixation pin (iMAP pin) 110, so antibiotic is transfused from the external transfusion tube 200 via the transfusion tube connector 120.

The method for connecting the transfusion tube connector 120 and the external transfusion tube 200 is not limited. For example, an adapter or socket may be employed. If the outer diameter of the one element can fit to the inner diameter of the other element, a plug-in connector can be employed and fixed by wrapping around with sealing tape. It is preferable to treat these elements carefully so as not to allow bacteria to access from this connecting portion because antibiotics are transfused through these elements.

In this example, the shape of the transfusion tube connector 120 is the shape that can fit to the lock connector 210 on the head portion of the external transfusion tube 200.

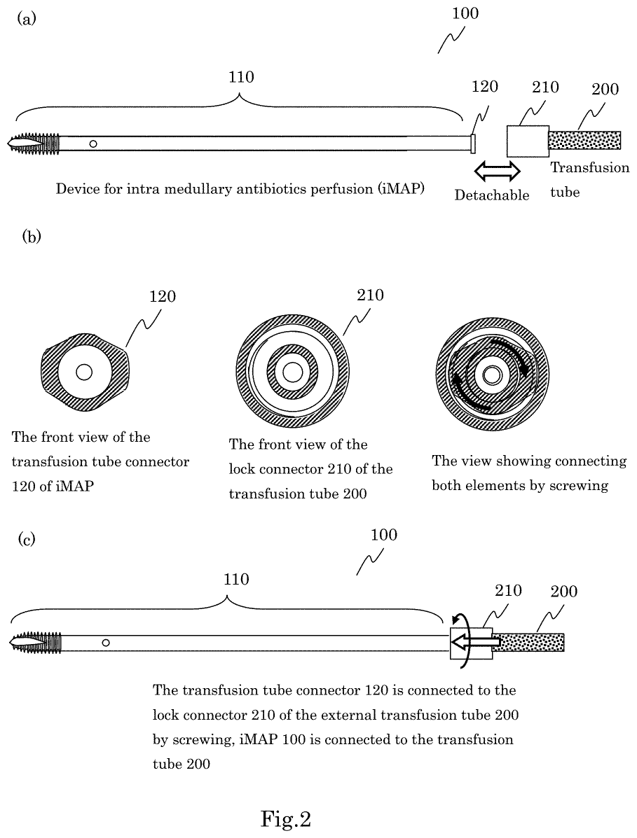

FIG. 2 is a schematic view showing the lock connector 210 of the external transfusion tube 200 is connected to the transfusion tube connector 120 of the iMAP 100 of Embodiment 1.

As shown in FIG. 2, the transfusion tube connector 120 of the iMAP 100 is connected to the lock connector 210 of the external transfusion tube 200 filled with antibiotics. Antibiotics are transfused from the external transfusion tube 200 to the base end opening 115 of the fixation pin (iMAP pin) 110 and the antibiotics are perfused from the side face opening 114 via the hollow portion 113.

As shown in FIG. 2, the transfusion tube connector 120 includes the male screw which fit to the inner female screw of the lock connector 210. The transfusion tube connector 120 can be connected to the lock connector 210 by screwing; the fixation pin (iMAP pin) 110 and the external transfusion tube 200 can be connected firmly.

Next, the procedure for connecting the drill attachment 140 in the electric drill connecting phase is described below.

The drill attachment 140 is an attachment for providing the element that can connect to a drill chuck 310 of an electric drill tool 300.

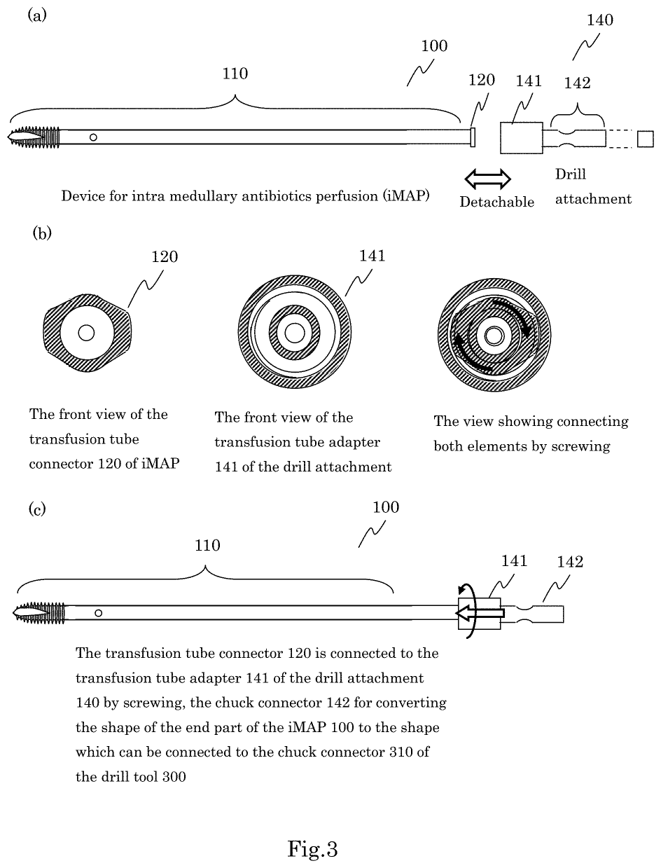

FIG. 3 is a schematic view of the structure of the drill attachment 140 and view showing the drill attachment 140 is connected to the transfusion tube adapter 141 of the iMAP 100 of Embodiment 1.

As shown in FIG. 3, the drill attachment 140 comprises a transfusion tube adapter 141 and a chuck connecter 142.

The transfusion tube adapter 141 has the same shape as that of the lock connector 210, so it can connect to the transfusion tube connector 120 on the base end part of the fixation pin (iMAP pin) 110.

As shown in FIG. 3 (b) and FIG. 3 (c), the transfusion tube connector 120 includes the male screw that fits to the inner female screw of the transfusion tube adapter 141. The transfusion tube connector 120 can be connected to the transfusion tube adapter 141 by screwing. As a result, the fixation pin (iMAP pin) 110 and the drill attachment 140 can be connected firmly.

When the drill attachment 140 is connected to the fixation pin (iMAP pin) 110, a chuck connecter 142 is provided at the end of the fixation pin (iMAP pin) 110. Therefore, the shape of the base end of the iMAP can converted to the shape that can connect to the drill chuck 310 of an electric drill tool 300.

The chuck connecter 142 is an element having the shape that can be chucked by the drill chuck 310 of the electric drill tool 300. The shape of the chuck connecter 142 is not limit to a particular shape as long as it can fit to the drill chuck 310 of the electric drill tool 300. In this example, the chuck connecter 142 includes an element whose cross sectional shape is a predetermined size square shape fit to the drill chuck 310; and the dimple portion in the inner wall that can accept a ball installed to the drill chuck 310 of an electric drill tool 300.

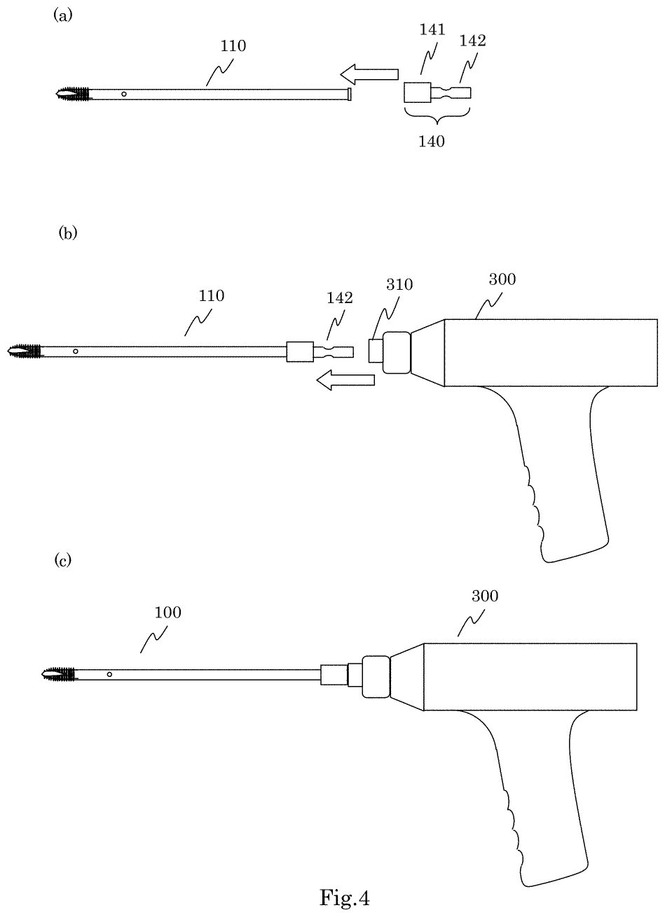

Next, the operation procedure of the iMAP 100 of Embodiment 1 is descried below with referencing to FIG. 4 to FIG. 6.

As shown in FIG. 4 (a), the drill attachment 140 is attached to the base end portion of the fixation pin (iMAP pin) 110 for preparing the electric drill connection phase as shown in FIG. 3.

As shown in FIG. 4 (b) to FIG. 4 (c), the drill chuck 310 of an electric drill tool 300 is connected to the chuck connecter 142 attached to the base end portion of the iMAP 100. The external electric drill tool 300 is connected to the iMAP 100.

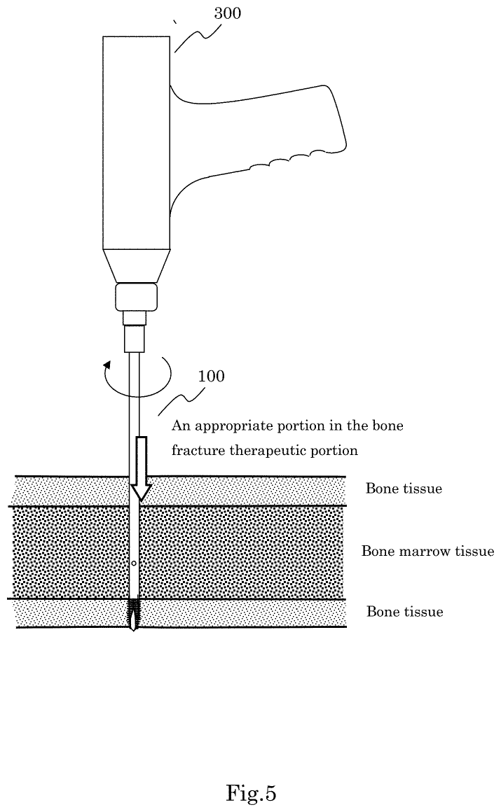

Next, As shown in FIG. 5, the iMAP 100 connected to the electric drill 300 of Embodiment 1 is fixed by piercing to the bone adjacent to and besides the patient's bone fracture surgical site. In this Embodiment 1, an operation for a single iMAP 100 is described. An operation for a plural set of iMAP 100 is described in Embodiment 3.

The fixation pin (iMAP pin) 110 has male screw 112 on the head portion for screwing. Beforepiercing the fixation pin (iMAP pin) 110 to the target bone portion, it is preferable that an assist hole that assists the beginning of screwing is opened beforehand by the electric drill tool. This assist hole can assist the fixation pin being screwed and piercing even if the target bone portion is hard bone tissue. The size of the assist hole should be adjusted to fit the fixation pin.

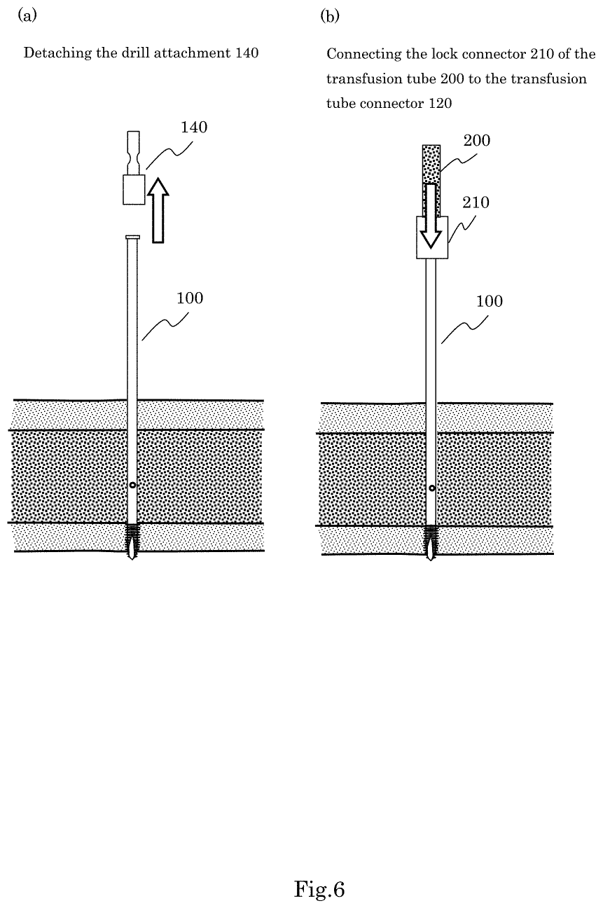

After fixing the fixation pin (iMAP pin) 110 to the appropriate portion according to the same operation as that of the external skeletal fixator, the electric drill tool and the drill attachment 140 are detached as shown in FIG. 6 (a). As a result, the iMAP 100 is left in the patient's bone fracture surgical site. In this state, the side face opening 114 is adjusted to face to the marrow of the patient's bone fracture surgical site.

A transfusion bag containing an appropriate kind and dose level of antibiotics is prepared at the appropriate timing during operation; the transfusion tube 200 is connected to the transfusion tube connector 120 via the lock connector 210 as shown in FIG. 6 (b).

An appropriate kind and dose level of antibiotics is perfused to the patient's bone fracture surgical site for a predetermined time and at a predetermined rate via the route from the transfusion tube 200—the lock connector 210—base end opening 115—the hollow portion 113—the side face opening 114.

The basic structure and function of the iMAP 100 and the each element are described as shown above. The iMAP 100 of the present invention is applied to the patient's bone fracture surgical site, and it is preferable that a required sterilization should be carried out on the iMAP as a medical use device.

### Embodiment 2

The device for intra medullary antibiotics perfusion (iMAP) 100a of embodiment 2 according to the present invention is described with reference to the drawings.

The iMAP has two phases for connecting to the external tools. The first phase is an electric drill connecting phase and the second phase is a transfusion tube connecting phase. In the configuration of Embodiment 2, the drill connector 150 used in the electric drill connecting phase is provided as a constant element, and the transfusion tube attachment 160 used in the transfusion tube connecting phase is provided as an attachment.

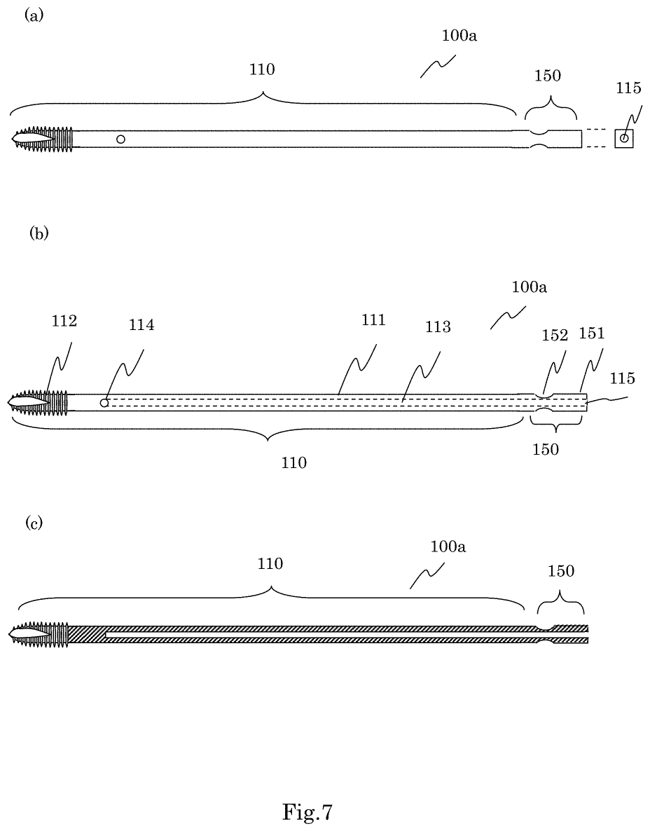

FIG. 7 is a schematic view of the structure of the device for intra medullary antibiotics perfusion (iMAP) 100a of this Embodiment 2.

As shown in FIG. 7 (a), the iMAP 100a includes a fixation pin (iMAP pin) 110, and a drill connector 150. FIG. 7 (b) is a figure for describing the elements of the iMAP 100a. In FIG. 7 (b), a hollow portion 113 as an inner structure is shown by broken lines to show the inner structure easily.

In this Embodiment 2, the fixation pin (iMAP pin) 110 and the drill connector 150 are formed as one body and cannot be separated from each other.

As shown in FIG. 7, the fixation pin (iMAP pin) 110 comprises a shaft 111, a screw part 112, a hollow portion 113, a side face opening 114 and a base end opening 115.

Regarding the shaft 111, the screw part 112, the hollow portion 113, and the side face opening 114 are the same as shown in Embodiment 1, so the descriptions for those are omitted here.

Antibiotics are delivered through the hollow portion 113. Therefore, the hollow portion 113 should penetrate to the base end opening 115 for introducing the transfused antibiotics. As shown in FIG. 7 (c), in this Embodiment 2, the drill connector 150 is formed on the base end portion of the fixation pin (iMAP pin) 110. Therefore the hollow portion 113 penetrates through the drill connector 150, and the base end opening 115 is installed in the end portion of the drill connector 150. From a different point of view, the drill connector 150 has a hollow, and this hollow is connected through the hollow portion 113 in the fixation pin (iMAP pin) 110.

It is preferable that the element of the fixation pin (iMAP pin) 110 is made from stainless steel or titanium metal, and the surface treatment with a hydroxyapatite coating is carried out.

The connecting operation of the lock connector 210 of the external transfusion tube 200 and the transfusion tube connector 120 in the antibiotic perfusion phase of the iMAP 100a of Embodiment 2 is described below.

The transfusion tube attachment 160 is an attachment for providing a structure for connecting to the lock connector 210. The transfusion tube attachment 160 can convert the shape of the base end part of the iMAP 110a to the shape applicable to the lock connector 120 on the head part of the transfusion tube 200.

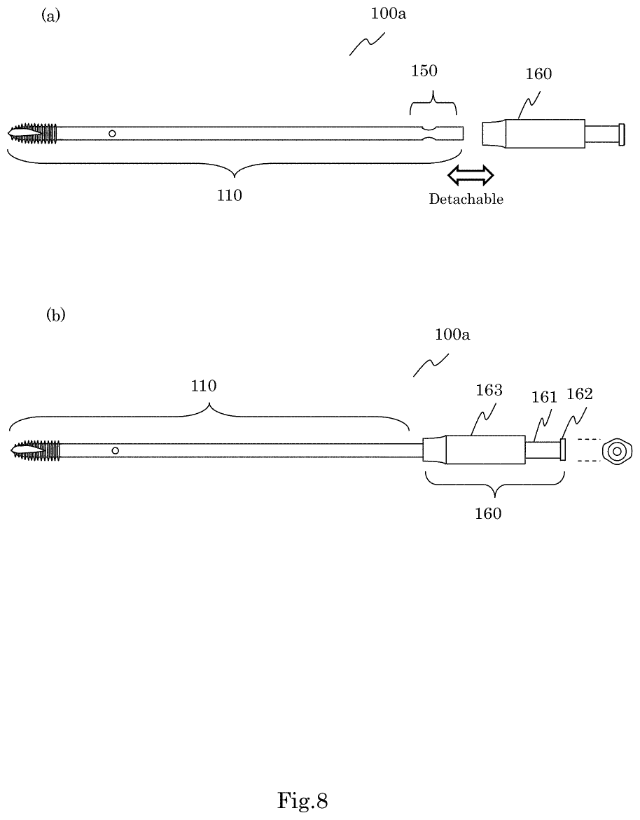

FIG. 8 is a schematic view showing the transfusion tube attachment 160 connected to the drill connector 150 of iMAP 100a of Embodiment 2. As shown in FIG. 8, the transfusion tube attachment 160 comprises a drill connector adapter 161 and a transfusion tube connector 162.

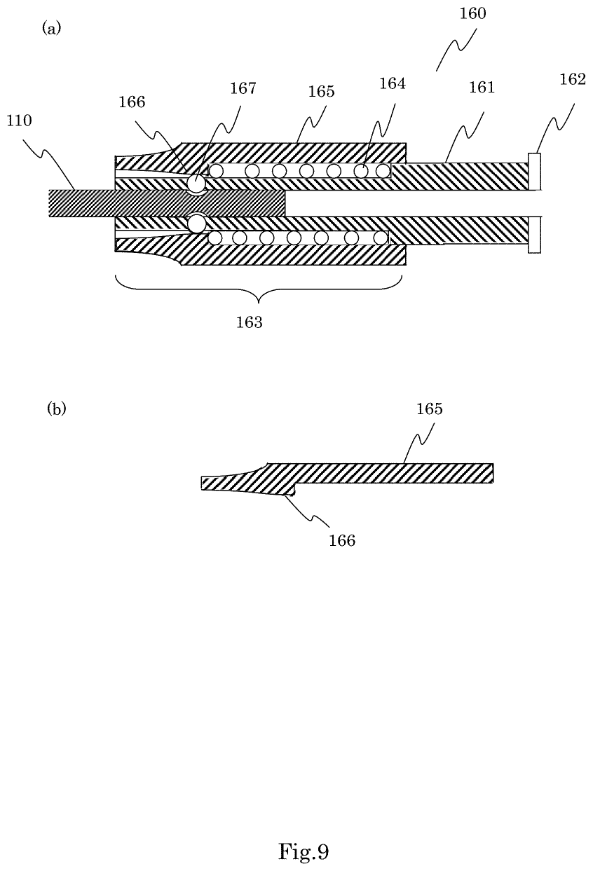

FIG. 9 is a schematic view of the structure of the transfusion tube attachment 160. It is an enlarged cross-sectional drawing in the longitudinal section to show the inner structure.

FIG. 9 (b) is a schematic view extracting the upper portion of a clipping force adjuster 165 to show a taper structure 166.

As shown in FIG. 8 and FIG. 9, the transfusion tube attachment 160 comprises the drill connector adapter 161 and the transfusion tube connector 162 and the detachable mechanism 163. In this example, the detachable mechanism 163 comprises an elastic actuator 164; a gripping force adjuster 165; a taper structure 166; and a gripping lock ball 167.

The drill connector adapter 161 is an adapter for connecting to the drill connector 150. The shape of the drill connector adapter 161 is the same as that of the drill chuck 310 of an electric drill tool 300. The drill connector 150 originally has the shape fitting to the drill chuck 310 of an electric drill tool 300, so the drill connector 150 can fit to the drill connector adapter 161 whose shape is the same as that of the drill chuck 310.

The transfusion tube connector 162 has a shape for connecting the lock connector 210 of the transfusion tube 200. The connection method for connecting the transfusion tube connector 162 and the transfusion tube 200 is not limited. In this Embodiment 2, the transfusion tube connector 162 is the same as that of Embodiment 1.

The detachable mechanism 163 can connect and detach the transfusion tube attachment 160 and the drill connector 150 formed on the fixation pin (iMAP pin) 110. Any mechanism can be employed as long as it can connect and detach the transfusion tube attachment 160 and the drill connector 150 as the detachable mechanism 163.

In this example, the detachable mechanism 163 adjusts the gripping force corresponding to the pressure generated between the gripping lock ball 167 and the outer surface of the drill connector 150. When the gripping force to the drill connector 150 by the gripping lock ball 167 becomes large, the transfusion tube attachment 160 is pressed to the drill connector 150. When the gripping force to the drill connector 150 by the gripping lock ball 167 becomes small, the transfusion tube attachment 160 is released from the drill connector 150 and detached.

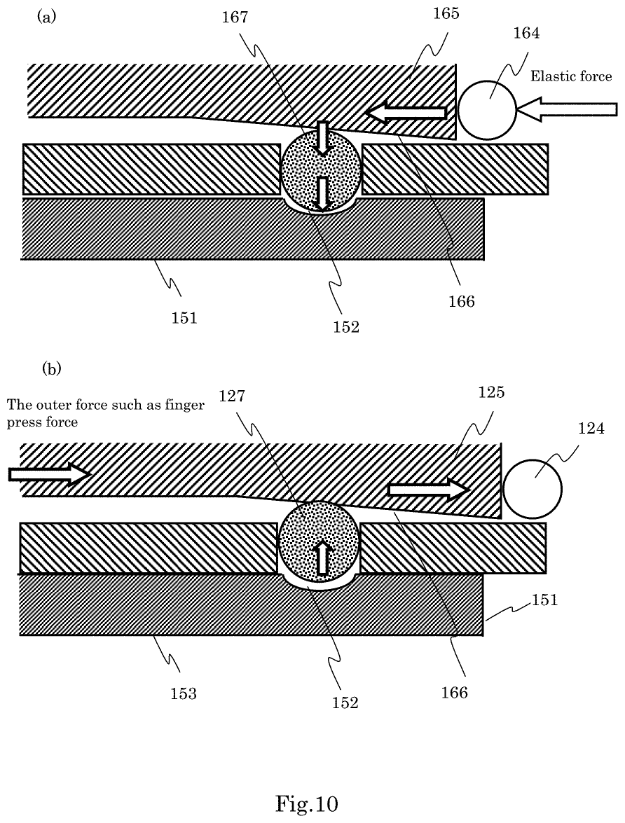

FIG. 10 is a schematic view showing the operation of the detachable mechanism, especially focusing on the operation of the gripping force adjuster 165 and the taper structure 166.

As shown in FIG. 10, the detachable mechanism 163 includes the gripping lock ball 167.

The gripping lock ball 167 is installed facing to the outer surface of the fixation pin (iMAP pin) 110. As shown in FIG. 10, a dimple portion 152 is formed on the inner surface of the drill connector 150 facing to the gripping lock ball 167.

By this configuration, the gripping force becomes larger when the gripping lock ball 167 is pressed stronger to the dimple portion 152. As shown in FIG. 8 (b), the transfusion tube attachment 160 is gripped to the drill connector 150 via the gripping lock ball 167 as one body.

The gripping force becomes smaller when the gripping lock ball 167 is pressed weaker to the dimple portion 152. When the gripping force is not enough to grip the transfusion tube attachment 160, the transfusion tube attachment 160 is detached from the fixation pin (iMAP pin) 110.

The detachable mechanism 163 adjusts the gripping force of the gripping lock ball 167. In this configuration, as shown in FIG. 9, the detachable mechanism 163 comprises the elastic actuator 164; the gripping force adjuster 165; the taper structure 166; and the gripping lock ball 167.

Any elastic mechanism such as a spring, an elastic material and a mechanism utilizing magnetic force can be employed as the elastic actuator 164. In this configuration, the inner space between the drill connector adapter 161 and the gripping force adjuster 165 is used for holding the elastic material for the stable operation.

The gripping force adjuster 165 is an element for sliding along by the elastic actuator 164. The gripping force adjuster 165 slides by pressing the gripping lock ball 167.

The sliding method for sliding the gripping force adjuster 165 is not limited. For example, the gripping force adjuster 165 is assembled with the drill connector adapter 161, the sliding range of the gripping force adjuster 165 is set along the drill connector adapter 161.

In this example, the taper structure 166 to press the gripping lock ball 167 is employed as a part of the gripping force adjuster 165. The taper structure 166 has a skew in that the head direction pushed by the elastic actuator 164 is thinner and the bottom portion attached to the gripping force adjuster 165 is thicker. Therefore, the taper structure 166 presses the gripping lock ball 167 down to the dimple portion 152 formed on the inner surface of the drill connector 150.

FIG. 10 is a schematic view showing the work of the gripping force adjuster 165 having the taper structure 166.

FIG. 10 (a) is a schematic view showing the work of the gripping force adjuster 165 sliding to the left in the drawing by the elastic force of the elastic actuator 164 and pressing down the gripping lock ball 167.

As shown in FIG. 10 (a), the elastic force of the elastic actuator 164 is applied on the gripping force adjuster 165 to slide to the left. The taper structure 166 of the gripping force adjuster 165 goes to the left, contacting the gripping lock ball 167 from the thinner portion. The gripping lock ball 167 is pressed down by the inner surface of the taper structure 166 (which is a part of the inner surface of the gripping force adjuster 165). The more the taper structure 166 goes to left, the larger the pressing force to the gripping lock ball 167 becomes. The gripping lock ball 167 also contacts on the dimple 152 in the electric drill connector 150, and the pressing force to the electric drill connector 150 by the gripping lock ball 167 becomes large. As a result, the friction between the electric drill connector 150 and the gripping lock ball 167 becomes large.

The transfusion tube attachment 160 is connected to the electric drill connector 150 via the gripping lock ball 167 and is held stably at an appropriate balancing position.

FIG. 10 (b) is a schematic view showing the work of the gripping force adjuster 165 sliding to the right in the drawing by the outer force such as finger press force against the elastic force of the elastic actuator 164 and releasing the gripping lock ball 167.

As shown in FIG. 10 (b), the outer force such as the finger press force is applied on the gripping force adjuster 165 to slide to the right. If the outer force overcomes the elastic force of the elastic actuator 164 and is enough for sliding the gripping force adjuster 165 to the right, the gripping force adjuster 165 begins to slide to the right. Then, the taper structure 166 of the gripping force adjuster 165 goes to right. The pressing force to the gripping lock ball 167 by the inner surface of the taper structure 166 (which is a part of the inner surface of the gripping force adjuster 165) becomes small. The more the taper structure 166 goes to the right, the smaller the pressing force to the gripping lock ball 167 becomes. As a result, the grip force between the electric drill connector 150 and the gripping lock ball 167 becomes small, and the transfusion tube attachment 160 is detached from the electric drill connector 150.

By this reversible operation as shown by FIG. 10 (a) and FIG. 10 (b), the transfusion tube attachment 160 can attach to and detach from the electric drill connector 150.

Next, the attachment procedure for the lock connector 210 of the transfusion tube 200 is described. The lock connector 210 is attached after attaching the transfusion tube attachment 160.

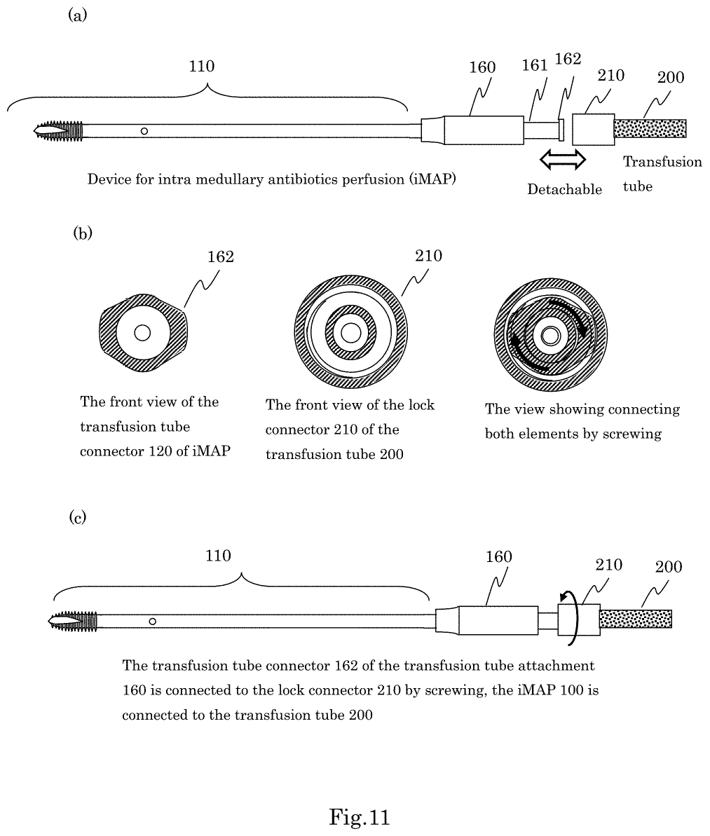

FIG. 11 is a schematic view showing the attachment procedure of the lock connector 210 of the transfusion tube 200 connecting to the transfusion tube connector 162 on the base end of the transfusion tube attachment 160.

In this example, the shape of the transfusion tube connector 162 is the same that of the transfusion tube connector 120 shown in Embodiment 1. As shown in FIG. 11, the same as FIG. 2, the shape of the transfusion tube connector 162 can fit to the inner female screw of the lock connector 210. Therefore, if the lock connector 210 screws into the transfusion tube connector 162, the lock connector 210 is connected to the transfusion tube connector 162. As a result, iMAP 100 and the transfusion tube 200 are connected each other.

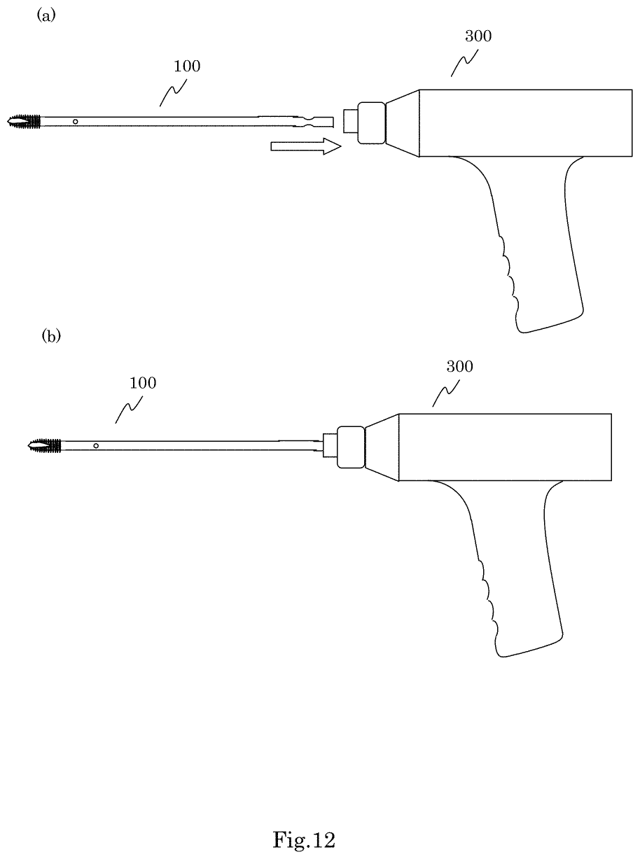

Next, the operation procedure of the iMAP 100a of Embodiment 2 is described below with reference to FIG. 12 to FIG. 14.

As shown in FIG. 12 (a), the drill chuck 310 of the electric drill tool 300 is attached to the base end portion of the fixation pin (iMAP pin) 110 of the iMAP 100a. As a result, the iMAP 100a is connected to the electric drill tool 300.

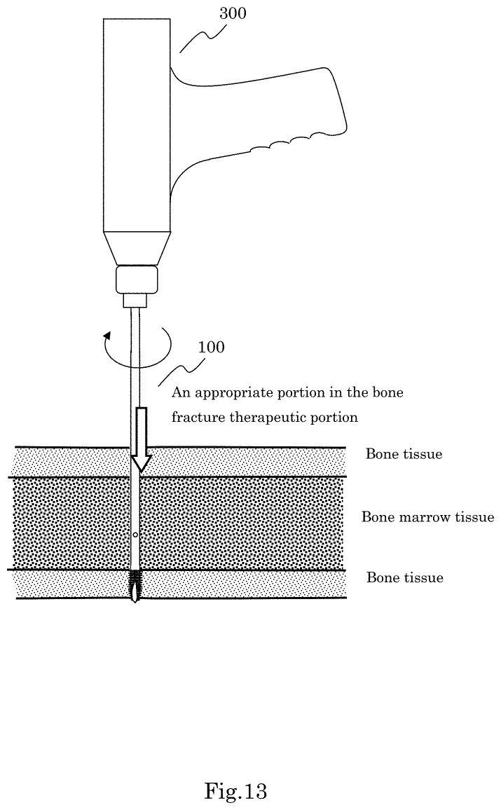

As shown in FIG. 13, the iMAP 100a of Embodiment 2 is pierced into the bone adjacent to and besides the patient's bone fracture surgical site. In this example, single iMAP 100a is fixed to the target bone. An operation for a plural set of iMAP 100 is described in Embodiment 3.

The fixation pin (iMAP pin) 110 has male screw 112 on the head portion for screwing. Before piercing the fixation pin (iMAP pin) 110 to the target bone portion, it is preferable that an assist hole which assists the beginning of screwing is opened beforehand by the electric drill tool. This assist hole can assist the fixation pin screwing and piercing even if the target bone portion is hard bone tissue. The size of the assist hole should be adjusted to fit the fixation pin.

After fixing the fixation pin (iMAP pin) 110 to the appropriate portion according to the same operation as that of the external skeletal fixator, the transfusion tube attachment 160 is connected to the drill connector 150. The procedure for connecting the transfusion tube attachment 160 to the drill connector 150 is shown in FIG. 10 (b). The gripping force adjuster 165 of the transfusion tube attachment 160 is pressed to the direction that the taper structure 166 is exit from the gripping lock ball 167 by a finger (the right direction in the drawing). The transfusion tube attachment 160 is inserted from the head portion to the drill connector 150 when the grip force given by the gripping lock ball 167 is small. After an appropriate insertion, the finger is released, then the gripping force adjuster 165 slides in the direction that the taper structure 166 goes into the gripping lock ball 167 by the elastic force of the elastic actuator 164. As a result, the grip force of the gripping lock ball 167 to the dimple 152 becomes large. The drill connector 150 is gripped by the gripping lock ball 167, and the transfusion tube attachment 160 can be attached firmly to the fixation pin (iMAP pin) 110.

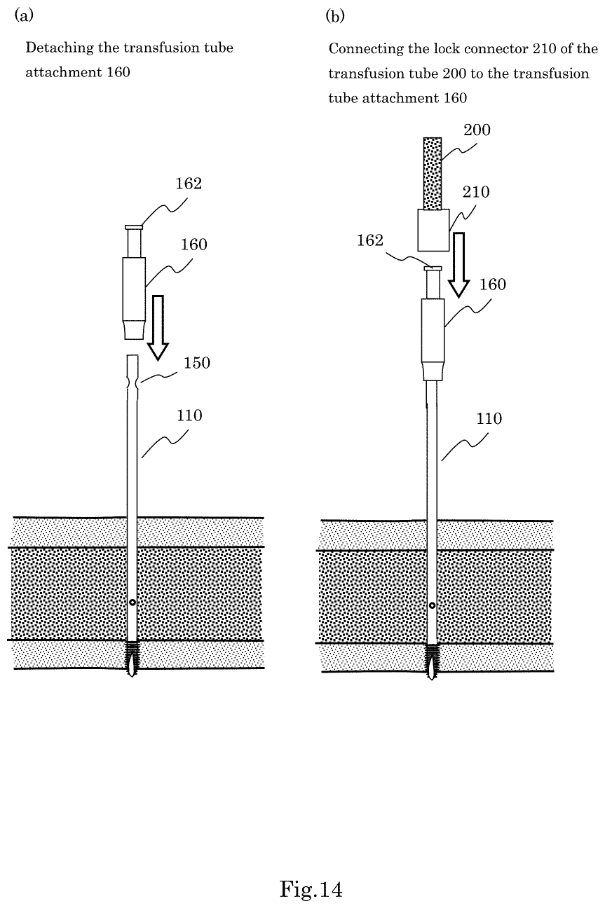

Next, as shown in FIG. 14 (b), the lock connector 210 screws into the transfusion tube connector 162, and the lock connector 210 is connected to the transfusion tube connector 162. As a result, iMAP 100 and the transfusion tube 200 are connected each other.

The appropriate kind and dose level of antibiotics is perfused to the patient's bone fracture surgical site for a predetermined time and at a predetermined rate.

The basic structure and function of the iMAP 100a and the each element of Embodiment 2 are described as shown above. The iMAP 100a of the present invention can be applied to the patient's bone fracture surgical site. Therefore, it is preferable that a required sterilization be carried out on the iMAP as a medical use device.

### Embodiment 3

The device for intra medullary antibiotics perfusion (iMAP) 100 of embodiment 3 according to the present invention is used as both an iMAP and a conventional external fixator to the patient's bone fracture surgical site.

In this example, the material and the shape of the shaft 111 and the screw part 112 of the fixation pin (iMAP pin) 110 of the iMAP 100 are the same as that of the conventional external fixator.

As shown in Embodiment 1 and Embodiment 2, the conventional fixation pin of the external fixator is known in the orthopedics field. The conventional external fixator is a well-known medical tool that is a standard tool used in an external fixation operation for piercing into a bone adjacent to and besides the patient's bone fracture surgical site and fixing externally. It is said that the conventional external fixator has no problem in the orthopedics field and many valuable insights are accumulated.

The inventor Akihiro MARUO found that the fixation pin (iMAP pin) 110 of the present invention is the same in view of the piercing and fixing to the bone adjacent to and besides the patient's bone fracture surgical site as the conventional external fixator. Therefore, he thought the fixation pin (iMAP pin) 110 of the present invention can be provided as the conventional external fixator.

The inventor Akihiro MARUO took care that the outer diameter of the external fixator is not standardized completely yet, the bridge tool does not always correspond to all external fixators. The inventor Akihiro MARUO has invented that the fixation pin (iMAP pin) 110 of the iMAP 100 of the present invention that employs different parts along the axis whose diameter sizes are different.

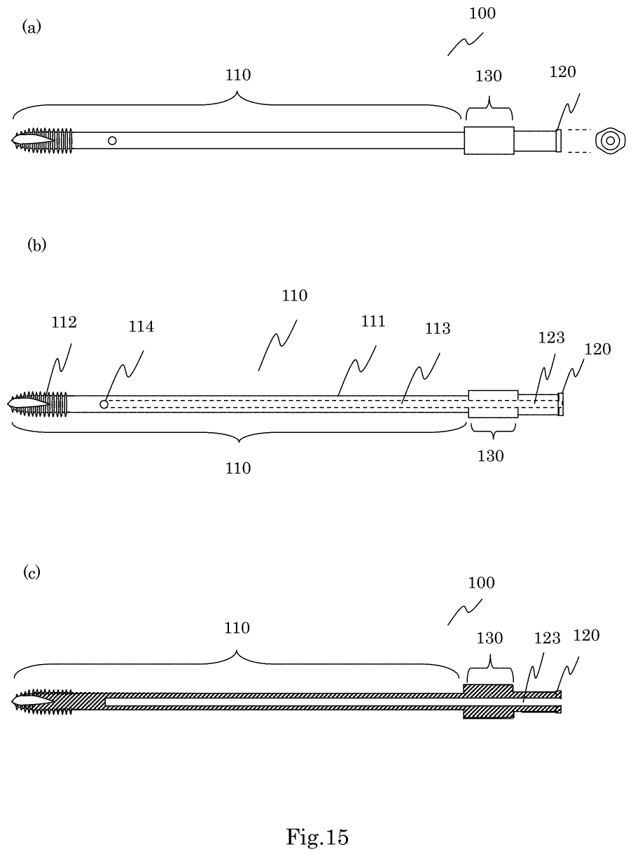

FIG. 15 is a schematic structure of the iMAP 100 of Embodiment 1 of the present invention which employs a different part 130 having a different diameter. As shown in FIG. 15, the outer diameter of the part 130 is larger than that of the other portion of the shaft 111. In short, the diameter R2 of the part 130 is larger than the diameter R1 of the shaft 111. A single iMAP 100 can provide both diameter R1 and diameter R2. Therefore, iMAP 100 can accept various bridge tools, and a variety of combinations of external fixator clusters can be formed.

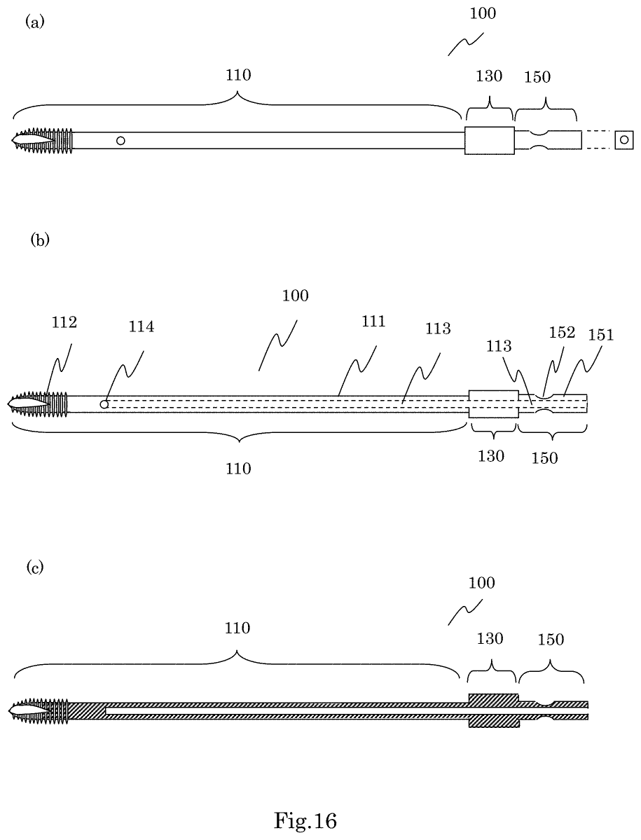

FIG. 16 is a schematic structure of the iMAP 100a of Embodiment 2 of the present invention which employs a different part 130 having a different diameter. The same as shown in FIG. 15, the outer diameter of the part 130 is larger than that of the other portion of the shaft 111. In short, the diameter R2 of the part 130 is larger than the diameter R1 of the shaft 111. A single iMAP 100 can provides both diameter R1 and diameter R2. Therefore, iMAP 100 can accept various bridge tools, and a variety of combinations of external fixator clusters can be formed.

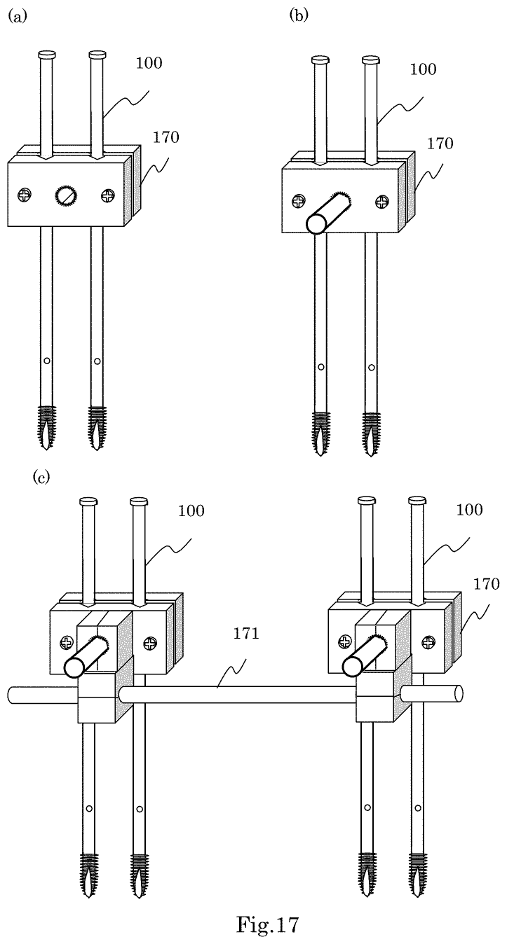

FIG. 17 is a schematic view showing the iMAP 100 of embodiment 3 is used as both an iMAP and a conventional external fixator to the patient's bone fracture surgical site. In this example, a plurality of iMAP 100 and the bridge tool 170 that bridges between iMAP 100 are employed.

As shown in FIG. 17, plural iMAP 100 are pierced and fixed to the bones including the trunk side and the end side of limbs adjacent to and besides the patient's bone fracture surgical site as the external fixator. For example, the case in which the bone fracture site is an open tibia fracture is described below. For example, a knee portion is selected for the trunk side and an ankle portion is selected for the end side of limbs as an appropriate portion for the tibia fracture surgical site. Plural iMAP 100 are piercing and fixing to those portions. Before piercing the fixation pin (iMAP pin) 110 to the target bone portion, it is preferable that an assist hole that assists the beginning of screwing is opened beforehand by the electric drill tool.

As shown in FIG. 17 (a), two iMAP 100 are pierced to the target portion each other. Then the bridge tool 170 connects between the fixation pin (iMAP pin) 110 of these iMAP 100 for stability. As shown above, plural iMAP 100 are connected via the bridge tool 170 as the cluster of the iMAP 100. The cluster of the iMAP 100 is more stable as a whole than the isolated single iMAP 100. The merits of preventing the iMAP 100 from tilting and being pulled out are obtained.

FIG. 17 (b) and FIG. 17 (c) show more complex patterns of the cluster of the iMAP 100. It is possible to bridge between bridge tools 170 with shaft tool 171.

The cluster of the iMAP 100 shown in FIG. 17 (c) is a state for providing a scaffold at the desired location according to the instruction of the doctor in charge, for perfusing antibiotics around the bone fracture surgical site.

The state shown in FIG. 17 (c) is also a state in which the external fixator fixes the bone fracture site externally. Therefore, an operation for the external fixing for the bone fracture site externally and an operation for combining a scaffold at the bone fracture site are done simultaneously.

A transfusion tube 200 is connected to the base end of an iMAP 100 or plural iMAP 100 selected among a cluster of the iMAP 100. An appropriate kind and dose level are selected, and antibiotics can be provided at necessary timing, for a desirable time and desirable rate via the transfusion tube 200 according to the circumstances of the tissue in the bone fracture therapeutic portion.

While some preferable embodiments of the sample storage according to the present invention are described above, it should be understood that various changes are possible, without deviating from the technical scope according to the present invention. Therefore, the technical scope according to the present invention is limited only by the claims attached.

### INDUSTRIAL APPLICABILITY

A device for intra medullary antibiotics perfusion according to the present invention can be employed as a device for intra medullary antibiotics perfusion for suppressing the occurrence of the post traumatic deep surgical site infection and various complications.

### DESCRIPTION OF THE REFERENCE NUMERALS

100: Device for intra medullary antibiotics perfusion

110: Fixation pin

111: Shaft

112: Screw part

113: Hollow portion

114: Side face opening

115: Base end opening

120: Transfusion tube connector

130: Different part having different diameter

140: Drill attachment

141: Transfusion tube adapter

142: Chuck connecter

150: Drill attachment

151: Body

152: Dimple portion

160: Transfusion tube attachment

161: Drill connector adapter

162: Transfusion tube connector

163: Detachable mechanism

164: Elastic actuator

165: Gripping force adjuster

166: Taper structure

167: Gripping lock ball

170: Bridge tool

1. A device for intra medullary antibiotics perfusion for perfusing an antibiotic to a patient's bone fracture surgical site, applied as an external skeletal fixator comprising;
a fixation pin for externally fixing to a bone by piercing from one side of cortical bone penetrating through bone marrow to the other side of cortical bone;
wherein the fixation pin includes a side face opening at the side of the shaft, which opening is located facing to the bone marrow; a base end opening at the base end part of the fixation pin; a hollow structure from the base end opening to the side face opening; and a male screw portion including spiral screw head mounted on the outer circumferential surface of the tip part of the fixation pin;
wherein a perfusion point of antibiotics is limited to the side face opening of the shaft, there is no point in the male screw portion of the tip of the fixation pin, and antibiotic is perfused only into the bone marrow via the side face opening of the shaft.

2. A device for intra medullary antibiotics perfusion applied as an external skeletal fixator according to claim 1, further comprising;
a transfusion tube connector installed to the end portion of the fixation pin, which includes an attachment structure for connecting to a lock connector attached to the head of the transfusion tube for antibiotics,
wherein the device for intra medullary antibiotics perfusion can connect directly to the transfusion tube for antibiotics via the transfusion tube connector.

3. A device for intra medullary antibiotics perfusion applied as an external skeletal fixator according to claim 2, further comprising;
a drill attachment for providing a structure connecting to a drill chuck of an electric drill tool comprising; a transfusion tube adapter for connecting to the transfusion tube connector on the base end part of the fixation pin, wherein the adapter shape is the same as that of the lock connector on the head part of the transfusion tube; a chuck connecter which can be chucked by the drill chuck of the electric drill tool;
wherein the shape of the base end part of the fixation pin can be converted to the shape applicable to the electric drill tool by the drill attachment.

4. A device for intra medullary antibiotics perfusion applied as an external skeletal fixator according to claim 1, further comprising;
a drill connector on the base end part, whose shape can be applicable to the electric drill tool, the fixation pin being capable of being chucked directly by the drill chuck of the electric drill tool.

5. A device for intra medullary antibiotics perfusion applied as an external skeletal fixator according to claim 4, further comprising;
a transfusion tube attachment for providing a structure for connecting to the lock connector installed to the head of the transfusion tube for antibiotics,
wherein the transfusion tube attachment comprises a drill connector adapter for connecting to the drill connector; and a transfusion tube connector for connecting to the lock connector on the head part of the transfusion tube;
wherein the shape of the base end part of the fixation pin can be converted to the shape applicable to the lock connector on the head part of the transfusion tube.

6. A device for intra medullary antibiotics perfusion applied as an external skeletal fixator according to claim 5, wherein the transfusion tube attachment includes a detachable mechanism which can adjust the gripping force between the inner surface of the drill connector adapter and the outer surface of the drill connector of the fixation pin,
wherein the detachable mechanism comprises a gripping lock ball installed facing to the outer surface of the drill connector of the fixation pin; an elastic actuator; a gripping force adjuster for adjusting the gripping force corresponding to the pressure generated between the gripping lock ball and the outer surface of the drill connector when sliding along a track by the elastic actuator; wherein the structure of the gripping force adjuster contacting to the gripping lock ball has a wedge-shape;
when the gripping force adjuster moves along the track by the elastic actuator, the gripping force to the drill connector of the fixation pin by the gripping lock ball becomes large by the operation of the wedge-shape to the gripping lock ball.

7. A device for intra medullary antibiotics perfusion applied as an external skeletal fixator according to claim 1, wherein the fixation pin has a wider portion whose diameter is larger than that of other portion.

8. A device for intra medullary antibiotics perfusion applied as an external skeletal fixator according to claim 1, further comprising a bridge connector for connecting between plural fixation pins,
wherein plural fixation pins are fixed to plural points adjacent to and besides patient's bone fracture surgical site and combined as a cluster of the devices for intra medullary antibiotics perfusion, wherein antibiotics agent is perfused from at least one of the devices for intra medullary antibiotics perfusion among the cluster.

9. A device for intra medullary antibiotics perfusion applied as an external skeletal fixator according to claim 1, wherein the outer shape of the fixation pin is the same as the outer shape of a conventional pin for an external skeletal fixation applied to patient's bone fracture surgical site, and the device for intra medullary antibiotics perfusion can apply as the external skeletal fixator providing both external skeletal fixation function and intra medullary antibiotics perfusion function to patient's bone fracture surgical site.

10. A method for providing intra medullary antibiotic perfusion to a subject in need thereof, comprising implanting a device of claim 1 in a bone of the subject, and perfusing antibiotic to the bone of the subject from the device according to claim 1.
